(19) United States
(12) Patent Application Publication
(10) Pub. No.: US 2024/0366431 A1
(43) Pub. Date: Nov. 7, 2024

(54) BANDAGE SYSTEM FOR DETECTION OF STAPHYLOCOCCUS ARUEUS AND METHOD OF USE

(71) Applicant: Emma J. Bright, Liberty, MO (US)

(72) Inventor: Emma J. Bright, Liberty, MO (US)

(21) Appl. No.: 18/652,195

(22) Filed: May 1, 2024

Related U.S. Application Data
(60) Provisional application No. 63/463,095, filed on May 1, 2023.

(51) Int. Cl. A61F 13/00

(52) U.S. Cl. CPC A61F 13/00059

(57) ABSTRACT

A disposable adhesive bandage capable of identifying the presence of Staphylococcus aureus (“S. aureus”) at a wound site. By utilizing elements of existing rapid tests, a standard adhesive bandage can be upgraded to detect S. aureus or a proprietary bandage could be manufactured containing the necessary components for such detection without detracting from the bandage's primary capabilities of protecting and covering a wound site. In another embodiment, the bandage pad is manufactured to perform the steps of the testing surfaces.

### CROSS-REFERENCE TO RELATED APPLICATION

This application claims priority in U.S. Provisional Patent Application No. 63/463,095, Filed May 1, 2023, which is incorporated herein by reference.

### BACKGROUND OF THE INVENTION

### 1. Field of the Invention

The present invention relates generally to a bandage system and method for use thereof, and more specifically to a bandage configured to provide rapid detection of Staphylococcus aureus.

### 2. Description of the Related Art

Staphylococcus aureus (“S. aureus”) is a hazardous disease if it is not caught in time. Doctors identified over 119,000 people in the United States with an S. aureus infection in their bloodstream, and almost 20,000 citizens died due to this bacterium according to the CDC.

Several differences exist between S. aureus and other Staphylococcus variants or other bacteria. No one factor of S. aureus makes it different from all other bacteria worldwide. However, in routine laboratory practice, the production of coagulase from S. aureus is frequently used as the sole criterion to distinguish S. aureus from other staphylococci, and there is a coagulase test. S. aureus also produces several other products that can be tested, for instance, capsular polysaccharides, protein A, and a toxin. Not all, but some S. aureus strains have also been known to produce Panton-Valentine Leucocidin, toxic shock syndrome 1 toxin (TSST-1), or other toxins.

Studies indicate that one-billion people in the world, at any given time, could give someone else a deadly infection or get a deadly disease themself. S. aureus is relatively harmless on healthy skin. If S. aureus is allowed to enter the bloodstream or the internal tissue, it becomes a health risk by way of infection. The way that S. aureus enters the bloodstream is most often when a wound is not properly treated, and the surrounding bacteria (in this case S. aureus) can invade the injured.

There are currently no tests for rapid identification of S. aureus that are easily accessible to the general public, it is evident that something had to happen to change the process. S. aureus is one of the most common bacterial infections that lead to complications such as bloodstream infections, pneumonia, or bone and joint infections. One such system for detecting S. aureus is the Pastorex Staph Plus system provided by Bio-Rad Laboratories, Inc. of Hercules, CA. When using Bio-Rad Pastorex Staph Plus, the S. aureus must be grown in a petri dish from the infected wound. What is needed is a way to quickly and easily detect S. aureus by end users outside of a lab or hospital environment.

Heretofore there has not been available a system or method for a bandage with the advantages and features of the present invention.

### BRIEF SUMMARY OF THE INVENTION

The present invention generally provides a disposable adhesive bandage capable of identifying the presence of Staphylococcus aureus (“S. aureus”) at a wound site. By utilizing elements of existing rapid tests, a standard adhesive bandage can be upgraded to detect S. aureus or a proprietary bandage could be manufactured containing the necessary components for such detection without detracting from the bandage's primary capabilities of protecting and covering a wound site.

In a first embodiment, a standard bandage having a pair of adhesive wings bounding a central bandage pad forms the basis of the invention. A latex test testing strip and a negative control testing strip are placed onto or within the central bandage pad. A mesh top layer may be used to secure the test strips in place. Latex test drops and negative control drops may be required to activate the test strips which alert the user if an S. aureus infection is detected once placed about the wound site without falsely identifying other Staphylococcus or other infection types, including Staphylococcus epidermidis.

The test strips associated with Pastorex Staph Plus system provided by Bio-Rad Laboratories, Inc. of Hercules, CA shows clumping on the test strip when used with S. aureus coming into contact with the strip. In an alternative embodiment, the entire padded central area of the bandage could function as the S. aureus test site, increasing size and removing the need for additional steps of construction.

### BRIEF DESCRIPTION OF THE DRAWINGS

The drawings constitute a part of this specification and include exemplary embodiments of the present invention illustrating various objects and features thereof.

FIG. 1 is a three-dimensional isometric view of a preferred embodiment of the present invention shown in an assembled orientation.

FIG. 2 is a partially-exploded three-dimensional isometric view thereof.

FIG. 3 is a fully-exploded three-dimensional isometric view thereof.

FIG. 4 is a flowchart diagramming the steps taken in practicing a method of the present invention.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

### II. Preferred Embodiment Bandage System 2

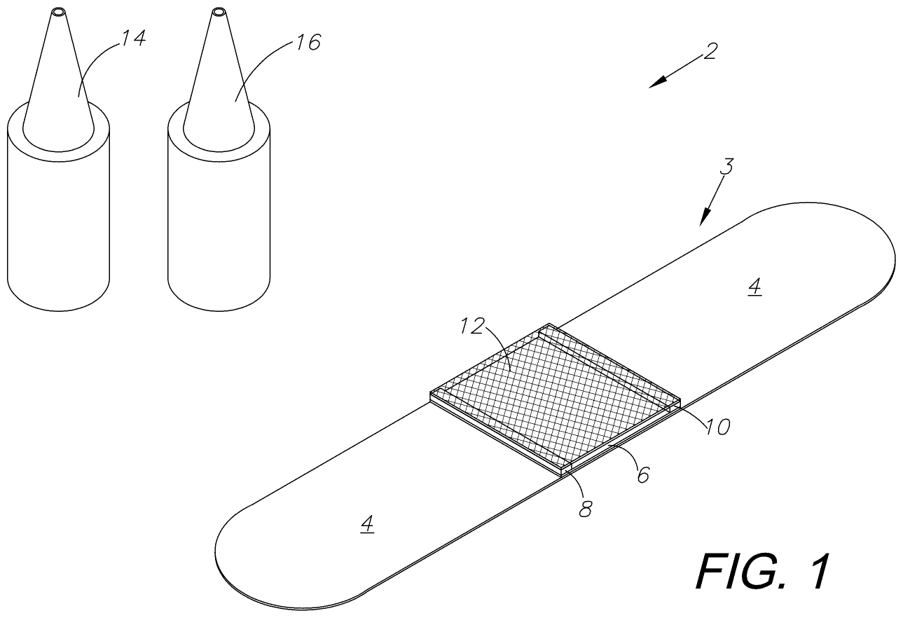

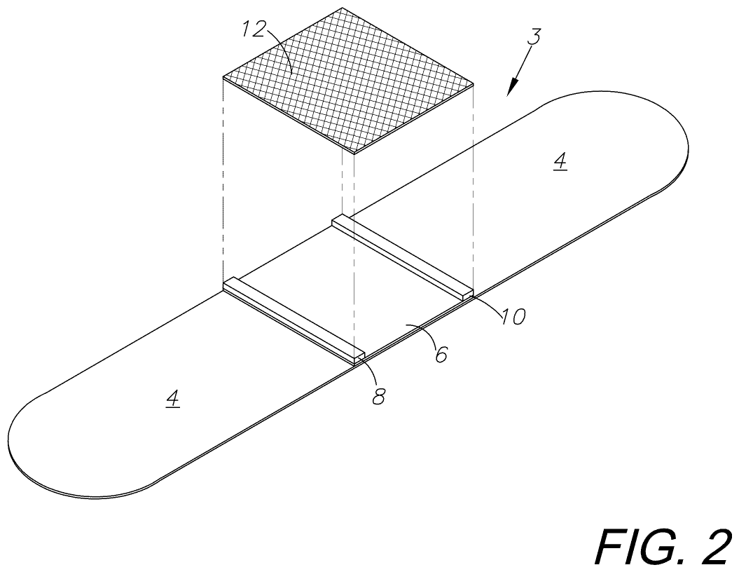

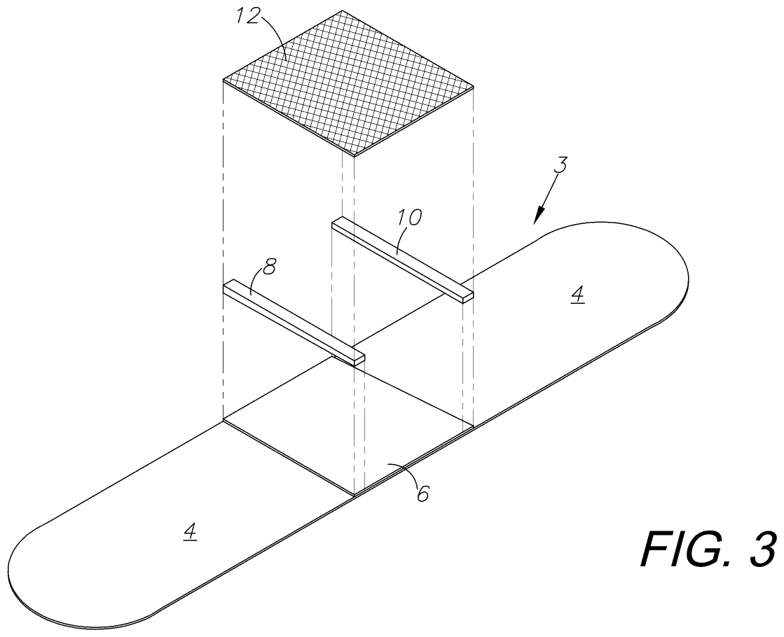

As shown in FIGS. 1-3, the present system teaches a bandage system 2 which is aimed at both securing a wound site while also testing that wound site to determine of any infections are present at the wound site. This system 2 could employ an existing bandage 3 which has a pair of adhesive-backed wings 4 and can include a central padded portion 6 typical of bandages, such as BAND-AID® adhesive bandages provided by Kenvue Inc. of Montgomery, NJ. This padded portion divides the bandage to have the pair of adhesive wings 4 on either side of the central padded portion 6.

A test surface 8 is included to detect whether Staphylococcus aureus (“S. aureus”) is present at a wound site. Other infection types can also be targeted. This test surface 8 could be integrated into the central padded portion 6 directly, or it may be present in the form of a test strip 8 either affixed to or inserted into the central padded portion.

A negative control surface 10 could also be included alongside the test surface. This negative control surface would be able to then act as a control case to reduce false positives or false negatives.

A preferred embodiment could function without additional steps once the bandage is placed at the wound site. If Staphylococcus aureus is located at the wound site, the test surface 8 will indicate such by displaying results of a positive test to the user. This can be in the form of clumping appearing at the test site, or the test surface could be modified to present a color-coded or otherwise obvious determination of a positive or negative result.

An embodiment may require liquid drops 14, 16 to be placed between the test surface and the wound site. These liquid drops would activate with the test surface and the S. aureus, thereby causing the bandage to indicate that S. aureus is present at the wound site. The liquid drops may include a first bottle 14 of drops for the test case surface 8 and a second bottle 16 of drops for the control case surface 10.

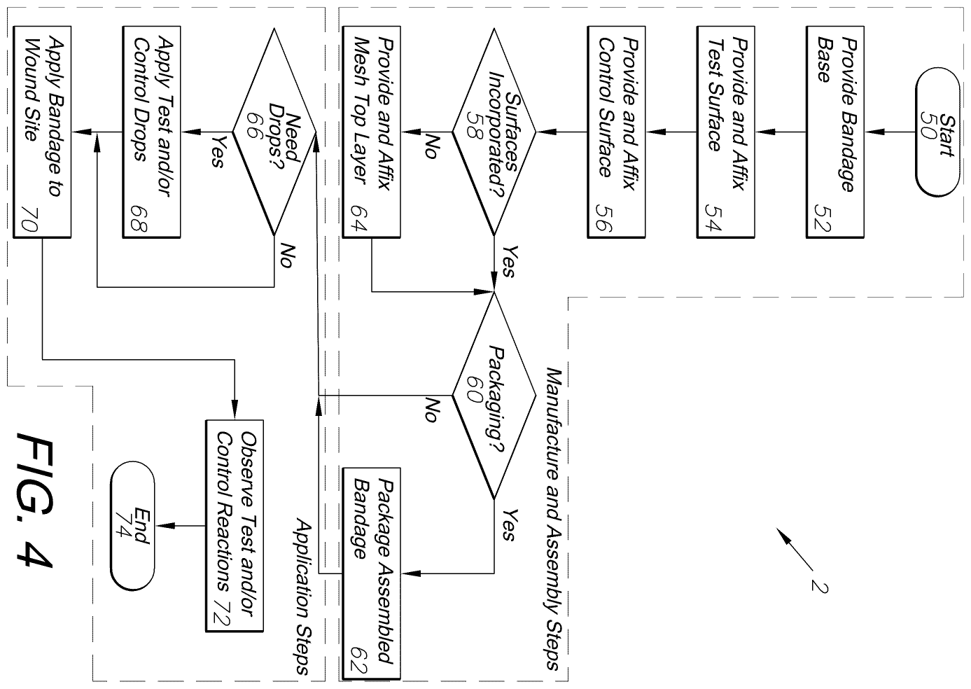

FIG. 4 steps through the method of manufacturing and practicing the Bandage System 2. The Manufacture and Assembly Steps are shown on the upper half of the diagram, with the steps taken for Application Steps at a wound site shown at the bottom half. The process begins at the “start” step at 50.

At 52, a bandage base 3 is provided. This could be a preexisting bandage or it could be a new bandage assembled for the purposes of the present invention. The components of the bandage are shown in FIGS. 1-3.

The test surface is provided and applied to the bandage at 54, and similarly the control surface is provided and applied at 56. If those test and control surfaces are incorporated into the bandage pad 6 at 58, then no additional steps are needed to assemble the bandage system 2. Otherwise, a mesh top layer 12 may be applied at 64. This helps to secure the test and control surfaces 8, 10 to the bandage 3.

A determination may be made at 60 whether the bandage system 2 requires packaging for future use, or if the bandage is going to be used immediately without packaging. If packaging is required, the assembled bandage is packed into its packaging at 62. Otherwise, the bandage may proceed to the Application Steps phase.

A determination is made at the outset of the Application Steps phase of the process whether the liquid drops 14, 16 are required at 66. This will depend on the bandage assembly process, whether the test 8 and control 10 surfaces are incorporated into the bandage pad 6, and whether those test 8 and control 10 surfaces require drops for results to appear. If yes, the drops are placed at 68 at the wound site or on the bandage pad 6 directly. Otherwise, the bandage is simply applied to the wound site at 70. The bandage is then observed at 72 to determine if positive results are returned from the test surface 8, which is compared with the control surface 10 to determine false negative results. The process ends at 74.

It is possible that a fully manufactured, stand-alone adhesive bandage could provide the benefits of the present invention, and may produce the best and fastest results to end-users. In such an instance, the bandage pad 6 element may partially or fully incorporate the capabilities of the test surface 8 and/or the control surface 10 such that they would replace the need for testing strips to be used.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. A bandage system comprising:
a central padded portion having a first side edge and a second side edge; and
a first flexible adhesive wing located along said first edge and a second flexible adhesive wing located along said second edge, said first and second flexible adhesive wings configured to secure said central padded portion about a wound site;
a test surface located within said central padded portions, said test surface configured to detect the existence of an infection when said test surface is placed in contact with said infection; and
said test surface further configured to visibly display positive results when said test surface is placed in contact with said wound site.

2. The system of claim 1, wherein said infection comprises Staphylococcus aureus (“S. aureus”).

3. The system of claim 1, further comprising:
a control surface located within said central padded portions, said control surface configured to provide feedback resulting in a false-positive result from said test surface; and
said control surface further configured to visibly display control results when said control surface is placed in contact with said wound site.

4. A method of manufacturing a bandage configured to detect an infection, the method comprising the steps:
providing a bandage having a pair of flexible wings comprising an adhesive face configured for adhering to a body surface, and said bandage further comprising a padded surface located between said pair of flexible wings, said padded surface configured for interfacing with a wound site within said body surface;
incorporating a test surface within said padded surface, said test surface configured to detect the presence of an infection at said wound site; and
producing a color response within said test surface as a result of detecting said infection at said wound site.

5. The method of claim 4, wherein said infection comprises Staphylococcus aureus (“S. aureus”).

6. The method of claim 4, further comprising the steps:
incorporating a control surface located within said central padded portions, said control surface configured to provide feedback resulting in a false-positive result from said test surface; and
visibly displaying a reaction within said control surface based upon said control results when said control surface is placed in contact with said wound site.
